(19) United States
(12) Patent Application Publication
(10) Pub. No.: US 2020/0289479 A1
(43) Pub. Date: Sep. 17, 2020

(54) STABILIZED TRANSDERMAL DELIVERY SYSTEM

(71) Applicant: AMW GMBH, Warngau (DE)

(72) Inventors: Verena SONNTAG, Putzbrunn (DE); Martina KESSLER, München (DE); Hubert KAFFL, Fischbachau (DE)

(73) Assignee: AMW GMBH, Warngau (DE)

(21) Appl. No.: 16/757,317

(22) PCT Filed: Oct. 19, 2018
(86) PCT No.: PCT/EP2018/078734

§ 371 Date: Apr. 17, 2020

(30) Foreign Application Priority Data
Oct. 20, 2017 (DE) 10 2017 124 626.6
Aug. 22, 2018 (DE) 10 2018 120 506.6

(51) Int. Cl. A61K 31/439; A61K 9/70; A61K 47/44; A61K 47/02; A61K 47/32; A61K 31/381; A61K 31/485; A61K 31/4468

(52) U.S. Cl. CPC A61K 31/439; A61K 9/7084; A61K 47/44; A61K 31/4468; A61K 47/32; A61K 31/381; A61K 31/485; A61K 47/02

(57) ABSTRACT

The present invention concerns a method for the production of a transdermal delivery system (TDS) comprising at least one active ingredient, wherein the method comprises temperature-conditioning. Furthermore, the present invention concerns a TDS which can be produced by means of the method in accordance with the invention, as well as the use of the TDS in accordance with the invention.

The present invention relates to a method for the production of a transdermal delivery system (TDS), comprising at least one active ingredient, wherein the method comprises temperature-conditioning . Furthermore, the present invention relates to a TDS which can be produced by means of the method in accordance with the invention, as well as to the use of the TDS in accordance with the invention.

The transdermal administration of pharmaceutically active ingredients is particularly appropriate if, following oral administration, a large proportion of the active ingredient is metabolized by the mucous membranes of the gastrointestinal tract or is retained by the liver (First Pass Effect) and/or if the active ingredient has a low plasma half-life. The transdermal delivery system (synonymous with a transdermal therapeutic system, TTS) is applied to the skin and releases the active ingredient, which latter is then reabsorbed by the skin. In this regard, transdermal administration requires that the delivery form employed should deliver the active ingredient in as uniform and as controlled a manner as possible over a long period of time.

In order to guarantee a sufficiently high plasma concentration or to have a desired therapeutic effect, in general, delivery rates for the active ingredient onto or through the skin which are as high as possible are aimed for. The delivery rate for the active ingredient on the one hand depends on the permeability properties of the skin for a relevant active ingredient and on the other hand on the concentration of the active ingredient in the matrix of the transdermal therapeutic system. The permeability properties of the skin can usually be improved by what are known as permeation enhancers. In order to improve the active ingredient delivery rate, it is therefore usual to increase the concentration of active ingredient in the active ingredient reservoir until the saturation concentration is reached or even exceeded in order, in this manner, to increase the thermodynamic activity of the active ingredient.

However, such an increased saturation concentration has the consequence that during storage or during the period of application, because the saturation concentration is exceeded at least on occasion, recrystallization of the active ingredient in the active ingredient matrix can easily occur. Because of the recrystallization, the thermodynamic activity of the active ingredient decreases sharply and as a consequence, the delivery rate for the active ingredient is also reduced.

Furthermore, crystallization of the active ingredient in the active ingredient-containing matrix may be caused or initiated by an input of energy such as, for example, by pressure when rolling up the laminates, or during the punching process or because of crystallization nuclei.

A variety of systems have been proposed in the prior art, with which high concentrations of active ingredient can be obtained in the matrix of a patch, and at the same time the recrystallization of the active ingredient can be avoided.

As an example, from U.S. Pat. No. 4,624,665 A, a transdermal system is known which contains the active ingredient in a reservoir in a microencapsulated form. The construction and the production of that system is extremely complicated, because the active ingredient has to be microencapsulated and homogeneously distributed in a liquid phase which, in further operational steps, is embedded between the backing layer and membrane of the TTS.

EP 0 186 019 A1 describes active ingredient patches in which a polymer which is capable of swelling in water is added to a rubber/adhesive resin mass, from which the active ingredient can be released. However, it has been shown that the release of the active ingredient from those active ingredient patches is far too low and does not satisfy the therapeutic requirements.

DE 39 33 460 A1 describes active ingredient patches based on homopolymers and copolymers with at least one derivative of acrylic acid or methacrylic acid which additionally are intended to contain substances which can swell in water.

DE 195 00 662 A1 describes a transdermal therapeutic system with a reservoir of active ingredient based on ethyl cellulose with a large proportion of rosin esters as a tackifying resin which is intended to counteract recrystallization of the active ingredient and thus reduce its release rate.

Thus, a transdermal system is intended to be developed, the production of which on the one hand is innocuous for the active ingredient-containing matrix and in which crystallization nuclei and/or crystals can be brought into solution without being damaged but which, however, guarantees the best possible stability of the active ingredient and of the matrix.

It is therefore an objective of the present invention to prevent the crystallization of active ingredients in the active ingredient-containing matrix, in particular in transdermal therapeutic systems, but wherein however, a best possible stability of the active ingredient and of the matrix is made possible. Furthermore, an objective of the present invention is to provide a method for the production of TDS with an active ingredient-containing matrix, preferably on a non-aqueous basis, in order to prevent the crystallization of the active ingredient in the matrix without substantially compromising the stability of the active ingredient and of the matrix.

In accordance with the invention, this objective is achieved by means of a method for the production of a TDS with an active ingredient-containing matrix, preferably on a non-aqueous basis, in accordance with claim 1, as well as by means of a TDS in accordance with claim 4. Furthermore, the objective is achieved by means of a TDS in accordance with the invention for application in accordance with claim 12.

Thus, the present invention concerns a method for the production of a transdermal delivery system comprising the steps of:
(i) providing at least one active ingredient-containing matrix, preferably an active ingredient-containing matrix on a non-aqueous basis,
(ii) applying the at least one active ingredient-containing matrix to a film in order to obtain a laminate with an active ingredient-containing matrix,
(iii) optionally, drying the laminate comprising the active ingredient-containing matrix,
(iv) optionally, punching out transdermal therapeutic systems in order to obtain laminar active ingredient cores,
(v) optionally, packaging transdermal therapeutic systems,
(vi) temperature-conditioning the active ingredient-containing matrix and/or the laminate and/or the punched transdermal therapeutic systems and/or the packaged transdermal therapeutic systems,
wherein the temperature-conditioning is carried out directly, preferably within 7 days, preferably within three days, particularly preferably within two days, in particular within 24 hours following application of the at least one active ingredient-containing matrix to the film.

The term “providing” as used herein should be understood to mean a production on-site as well as a supply of an active ingredient-containing matrix, preferably an active ingredient-containing matrix on a non-aqueous basis. In this regard, an active ingredient-containing matrix may already be provided with a protective film covering the application side of the active ingredient-containing matrix, which protective film remains on the matrix during the rest of production, or optionally may be replaced in one or more production steps by an alternative protective film.

The term “temperature-conditioning” should be understood to mean heating of the active ingredient-containing matrix and/or the laminate and/or the punched transdermal therapeutic systems and/or the packaged transdermal therapeutic systems to a moderately high temperature which is well adapted to requirements, at which the active ingredient-containing matrix or the active ingredient per se does not change in a disadvantageous manner, for example does not undergo any substantial physico-chemical modification, and/or crystallization of the active ingredient does not occur. Preferably in this regard, temperature-conditioning of the punched transdermal therapeutic systems and/or the packaged transdermal therapeutic systems is carried out.

In accordance with a preferred embodiment, laminar active ingredient cores, for example matrix cores containing an active ingredient, may be punched out of the laminate with an active ingredient-containing matrix before or after application onto a protective film and undergo the production method in accordance with the invention subsequently. The term “active ingredient core” as used herein should be understood to mean a laminar active ingredient-containing matrix which advantageously comprises a backing layer or a protective film on its rear side and/or on its application side and which is cut, in particular punched, from the aforementioned laminate containing an active ingredient with the aid of a cutting tool, in particular a punching tool. Particularly preferably, laminar active ingredient cores are punched from a laminate which comprises the active ingredient-containing matrix preferably on a non-aqueous basis, and a protective film.

Furthermore, the present invention concerns a transdermal therapeutic system which is obtainable by the method described above.

Advantageously, the TDS which may be produced by the method in accordance with the invention may be stored at a preferred temperature of at least approximately 5° C. Preferably, the temperature for storage of the TDS in accordance with the invention is approximately 5° C. up to approximately 25° C. or a temperature of approximately 5° C. up to approximately 30° C. or a temperature of approximately 5° C. up to approximately 40° C., without a substantial proportion of the active ingredient crystallizing out and/or without the stability of the active ingredient and/or of the matrix being substantially compromised.

Thus, the composition in accordance with the invention or the TDS in accordance with the invention can advantageously be stored at room temperature for several months or even years, for example, without a substantial proportion of the active ingredient crystallizing out thereby or without the stability of the matrix being compromised, for example by cold flow. In particular, it has been observed that the formation of contaminants in the TDS which is produced by means of the method in accordance with the invention can be significantly reduced, or the stability of the active ingredient can be significantly improved.

Because of the aforementioned advantageous properties even after storage for several months or years, the TDS in accordance with the invention exhibits a reproducible and controllable delivery of the active ingredient onto or through the skin. This is particularly relevant for the use of the composition in accordance with the invention in forms of administration such as transdermal therapeutic systems in which a delivery of the active ingredient from a crystalline form is substantially delayed or reduced or when a sufficient amount of the active ingredient is not available because of deterioration of the active ingredient, and hence an effective therapy cannot be guaranteed.

The transdermal therapeutic system in accordance with the invention furthermore has a reduced moisture content, in particular a reduced water content, which is advantageous for the stability as regards recrystallization of the active ingredient.

Advantageously, therefore, the TDS in accordance with the invention has a water content in the dried matrix of up to approximately 2% by weight, preferably of up to approximately 1% by weight, particularly preferably of up to approximately 0.75% by weight, in particular of up to approximately 0.5% by weight, particularly preferably of up to approximately 0.2% by weight.

In its simplest embodiment, the transdermal therapeutic systems in accordance with the invention comprise a backing layer, at least one matrix layer which contains an active ingredient which is preferably non-aqueous, which is disposed horizontally in the TDS and with which the TTS is applied to the skin, as well as an optional protective layer which is applied to the active ingredient-containing matrix and is removed prior to application of the TTS.

The term “matrix on a non-aqueous basis” or “non-aqueous matrix” as used herein should be understood to mean a matrix or a plurality of matrices the constituent components of which, in particular their polymers, are basically non-aqueous. Preferably, the active ingredient-containing matrix layer on a non-aqueous basis is self-adhesive. Hereinbelow, the formulation “matrix on a non-aqueous basis” will be used synonymously with the formulation “non-aqueous matrix”.

However, the present invention also encompasses a TTS with a more complex construction such as, for example, with two or more matrix layers with different compositions and adhesive strength.

The side of the TDS which in general is intended to be placed on the skin or intended to come into contact with the skin is termed the “application side”. The application side in this regard may be configured so as to have pressure-sensitive adhesive properties over its entire surface, for example wherein the non-aqueous active ingredient-containing matrix itself has contact adhesive properties or is coated over its entire surface with a self-adhesive glue, or it may be configured so as to be adhesive over only a portion of its surface.

In principle, the transdermal therapeutic systems in accordance with the invention are suitable for the application of any active ingredients or combinations of active ingredients. In the context of the present invention, in this regard, the term “active ingredient” should be understood to mean a pharmaceutically effective substance or even a cosmetically active ingredient and/or an additive and/or a nutrient or a food supplement.

The transdermal therapeutic system in accordance with the invention delivers the active ingredient from the preferably non-aqueous active ingredient-containing matrix onto the skin, wherein at least a portion of the active ingredient can be taken up systemically. The transdermal therapeutic system can therefore also be used for the dermal delivery of an active ingredient such as, for example, for local anesthesia, for antibiotic treatment or for the treatment of benign or malignant skin conditions.

The active ingredients may be contained in the matrix in a variety of forms, depending on which form provides the optimal delivery properties for the active ingredient from the TDS or the matrix. In the case of pharmaceutically effective substances, these may be in the form of the free base or acid or in the form of salts, esters, hydrates or other pharmacologically acceptable derivatives, or as components of molecular complexes.

The absolute quantity of active ingredient contained in the patch in general determines the period of time over which a continuous supply of the active ingredient into or onto the organism can be maintained. Thus, loading of the matrix with active ingredients which is as high as possible is desirable when the application period for a patch is long, i.e. a few days up to one week. A transdermal therapeutic system in accordance with the invention is, however, preferably used for an application period of two to seven days, in particular for an application period of three days.

Finally, the present invention also encompasses the medical, veterinary and/or cosmetic use of the patch in accordance with the invention for the delivery of active ingredients onto and optionally through the skin of a human or animal body and/or onto an environment around the patch.

Further particularly advantageous embodiments and further embodiments of the invention will become apparent from the dependent claims as well as from the description below, wherein the patent claims of a specific category may also be construed in accordance with the independent claims of another category and features of various exemplary embodiments may be combined into new exemplary embodiments.

In accordance with a preferred method for the production of the transdermal delivery system, the temperature-conditioning of the active ingredient-containing matrix and/or of the laminate and/or of the punched transdermal therapeutic systems and/or of the packaged transdermal therapeutic systems is carried out at a temperature of at least approximately 50° C., preferably at a temperature of at least approximately 60° C., particularly preferably at a temperature of at least approximately 70° C. At most, temperature-conditioning in this regard is carried out at a temperature of up to approximately 120° C., preferably at a temperature of up to approximately 100° C., particularly preferably at a temperature of up to approximately 90° C., in particular at a temperature of up to approximately 80° C., particularly preferably at a temperature of approximately 75° C.

Preferably, temperature-conditioning in this regard is carried out over a time period of up to 12 hours, preferably over a time period of up to 6 hours, particularly preferably over a time period of up to 2.5 hours, in particular over a time period of up to approximately 1 hour, particularly preferably over a time period of up to approximately 30 minutes, more particularly preferably over a time period of up to approximately 15 minutes.

A protocol of this type advantageously enables complete dissolution of the active ingredient and effectively prevents the formation of crystallization nuclei, whereupon a recrystallization of the active ingredient can be prevented or at least delayed. In particular, temperature-conditioning of the punched transdermal therapeutic systems and/or of the packaged transdermal therapeutic systems of this type is carried out.

In principle, temperature-conditioning may be carried out in combination with or immediately after drying the active ingredient-containing matrix or a laminate comprising the active ingredient-containing matrix. However, temperature-conditioning may also take place entirely independently of drying or in more than one temperature-conditioning procedure immediately after drying. Preferably, temperature-conditioning takes place independently of drying of the active ingredient-containing matrix; in particular, the temperature-conditioning is carried out after drying the laminate comprising the active ingredient-containing matrix, of the punched active ingredient core and/or of the packaged transdermal therapeutic systems, in particular of the punched transdermal therapeutic systems and/or of the packaged transdermal therapeutic systems.

In accordance with a preferred embodiment, an advantageous transdermal delivery system which can be produced by means of the method in accordance with the invention furthermore has an additional application layer which is free from active ingredient or contains an active ingredient, but particularly preferably contains an active ingredient. Preferably, an application layer containing an active ingredient comprises a matrix on a non-aqueous basis which in particular has adhesive properties.

However, as mentioned above, not all of the layers of the transdermal therapeutic system in accordance with the invention have to contain an active ingredient, but in addition to the at least one or two matrix layers containing an active ingredient, preferably on a non-aqueous basis, the TDS may have one or more layer(s) which are free from active ingredient, which are preferably adhesive in configuration.

A matrix layer of an advantageous TDS, particularly preferably a second active ingredient-containing matrix, may be separated from the first matrix, advantageously by means of a control membrane applied between the first and the second matrix. Preferably, the first and the second matrix are matrices on a non-aqueous basis. Thus, for example, a first non-aqueous active ingredient-containing matrix facing the backing layer may be separated by means of a control membrane from a second application layer containing an active ingredient, which in turn is preferably self-adhesive in configuration.

In a corresponding preferred production method, a control membrane is introduced between the matrix and an application layer which may contain an active ingredient or be free from an active ingredient. A control membrane of this type may be hydrophobic or hydrophilic in configuration. In particular, however, a control membrane of this type is hydrophobic in configuration. Diffusion of the active ingredient herein is possible by means of pores in the control membrane; the pore diameter is preferably at least 0.05 μm, particularly preferably at least 0.075 μm, in particular at least 0.1 μm. A preferred control membrane has pores with a pore diameter of up to approximately 0.5 μm, particularly preferably of up to approximately 0.25 μm, in particular of up to approximately 0.2 μm.

In particular, a preferred control membrane is based on a polymer selected from the group comprising polyolefins, olefin copolymerisates, polyesters, copolyesters, polyamides, copolyamides, polyurethanes and the like. Examples of suitable materials which may be cited are polyesters, and of these polyethylene terephthalates in particular, as well as polycarbonates, polyolefins such as, for example, polyethylenes, polypropylenes or polybutylenes, polyethylene oxides, polyurethanes, polystyrenes, polyamides, polyimides, polyvinyl acetates, polyvinyl chlorides, polyvinylidene chlorides, copolymerisates such as acrylonitrile-butadiene-styrene terpolymers, or ethylene-vinyl acetate copolymerisates, for example. In this regard, a particularly preferred control membrane comprises a polypropylene and can be obtained, for example, from Azelis (Germany) under the trade name Celgard.

In a further preferred embodiment, at least one matrix, preferably a matrix on a non-aqueous basis, of a transdermal delivery system in accordance with the invention has a mineral oil content; particularly preferably, a second or further matrix, preferably on a non-aqueous basis, also comprises a mineral oil content, wherein the mineral oil content in the first and in the second or further matrix may be the same or different. In this manner, a transdermal delivery system in a first matrix may preferably have a mineral oil content of at least approximately 25% by weight and/or at most up to approximately 40% by weight and/or a second matrix may preferably have a mineral oil content of at least approximately 35% by weight and/or at most up to approximately 50% by weight. An example of a suitable mineral oil is commercially available under the trade name Klearol from Sonneborn (Netherlands).

As a further component, a preferred advantageous TDS may comprise at least one matrix which is preferably on a non-aqueous basis, preferably also a second matrix, preferably on a non-aqueous basis, with a silicon dioxide content. An advantageous silicon dioxide content in the first matrix, preferably also in the second matrix, in this regard is preferably at least approximately 1% by weight, particularly preferably at least approximately 2.5% by weight. At most, a preferred silicon dioxide content in the first matrix, preferably also in the second matrix, is up to approximately 10% by weight, particularly preferably up to approximately 7.5% by weight, in particular approximately 5% by weight. In particular in this regard, the silicon dioxide has hydrophilic properties. A silicon dioxide of this type is, for example, commercially available from Evonik Degussa (Germany) under the trade name Aerosil® 200 Pharma.

A further preferred component of the matrix which is preferably on a non-aqueous basis, comprises a polyvinylpyrrolidone, in particular a crospovidone (Ph.Eur. 7th edition Supplement 7.4, Type B) or copovidone which, for example, is produced by BASF (Germany) under the trade name Kollidon® CL-M or Kollidon® VA64 and can be obtained from BTC Europe (Germany).

The polymers or the polymer systems of the TDS in accordance with the invention forming the at least one matrix preferably on a non-aqueous basis may in principle be adhesive or non-adhesive in configuration. Preferably, however, the polymer or the polymer systems have adhesive properties.

As already discussed, the TDS which may be produced by the method in accordance with the invention comprises a polymer system or a polymer blend, preferably on a non-aqueous basis forming the matrix, and containing a pharmaceutically active ingredient. in this regard, the polymer or the polymer blend, preferably on a non-aqueous basis, forming the matrix is not fundamental to the present invention, however some polymers are more suitable than others. Examples of suitable polymers forming the matrix in this regard are polybutenes or polyisobutylenes, polyacrylates, polysiloxanes, block copolymers such as, for example, styrene-butadiene-styrene block copolymers, silicones and blends thereof.

A preferred TDS in this regard comprises at least one active ingredient-containing matrix comprising at least one polybutylene or polyisobutylene and blends thereof such as, for example, blends of polybutylenes or polyisobutylenes with different molecular weights (MW). This does not exclude the fact that a blend of this type could also comprise one or more polymers formed from polybutylene or polyisobutylene, which in particular have a molecular weight in the aforementioned range. Finally, a first and/or second polyisobutylene polymer may be partially or completely replaced by a first and/or second polybutene or polybutylene. Thus, a preferred blend of polybutylene polymer or polyisobutylene polymers has a first polyisobutylene with a molecular weight of at least approximately 20,000 g/mol and/or of at most approximately 100,000 g/mol and/or a second polyisobutylene with a molecular weight of at least approximately 500,000 g/mol and/or of at most approximately 3,500,000 g/mol.

In accordance with a particularly preferred embodiment, a polymer blend of this type comprises at least two polybutylene polymers or polyisobutylene polymers, wherein a first of the at least two polybutylene polymers or polyisobutylene polymers preferably has a molecular weight of at least approximately 30,000 g/mol, particularly preferably of at least 35,000 g/mol; at most, a first of the at least two polybutylene polymers or polyisobutylene polymers has a molecular weight of up to approximately 45,000 g/mol.

A second polybutylene polymer or polyisobutylene polymer in this regard preferably has a molecular weight of at least approximately 800,000 g/mol and/or of at most approximately 1,200,000 g/mol. The ratio of a first polybutylene polymer or polyisobutylene polymer and a second polybutylene polymer or polyisobutylene polymer in this regard is preferably at least approximately 0.75 to approximately 1, particularly preferably at least approximately 1 to approximately 1, in particular approximately 1.25 to approximately 1. At most, a preferred ratio is up to approximately 2 to approximately 1, particularly preferably up to approximately 1.5 to approximately 1, in particular up to approximately 1.4 to approximately 1. In particular, a TDS with a polymer blend of this type comprises an active ingredient from the antiemetics class, particularly preferably a tropane alkaloid, in particular scopolamine.

In accordance with a further preferred embodiment, a polymer blend preferably comprises at least two polybutylene polymers or polyisobutylene polymers, wherein a first of the at least two polybutylene polymers or polyisobutylene polymers preferably has a molecular weight of at least approximately 30,000 g/mol, particularly preferably of approximately 35,000 g/mol; at most, a first of the at least two polybutylene polymers or polyisobutylene polymers has a molecular weight of up to approximately 45,000 g/mol. A second polybutylene or polyisobutylene in this regard preferably has a molecular weight of at least approximately 800,000 g/mol and/or at most approximately 1,200,000 g/mol, in particular 1,000,000 g/mol. In accordance with a further preferred embodiment, the ratio of a first polyisobutylene polymer and/or polybutene polymer to a second polyisobutene polymer and/or polybutylene polymer is up to approximately 9 to approximately 0.1, particularly preferably up to approximately 7 to approximately 0.5, in particular up to approximately 6 to approximately 1. In particular, a TDS with a polymer blend of this type comprises an active ingredient from the class of dopamine agonists, particularly preferably from the class of D2 agonists and ergot alkaloid derivatives, in particular rotigotine, pramipexole, ropinirole, cabergoline and/or lisuride, particularly preferably rotigotine.

In order to prevent a migration of active ingredient out of the matrix on a non-aqueous basis, a preferred transdermal delivery system advantageously has an occlusive backing layer. Usually, what are known as backing films produced, for example, from polyesters with a thickness which is preferably at least approximately 5 μm, particularly preferably at least approximately 10 μm, in particular at least approximately 20 μm, particularly preferably at least approximately 30 μm, are used as the backing layer of a TDS in accordance with the invention. At most in this regard, a backing film produced from polyester, for example, has a thickness which is preferably up to approximately 200 μm, particularly preferably up to approximately 150 μm, in particular up to approximately 100 μm, particularly preferably up to approximately 50 μm, most preferably up to approximately 40 μm. Backing films of this type are flexible and can optionally be positioned around the edges of the matrix layer, i.e. around the side faces of the active ingredient-containing matrix orientated in the lateral direction, and cover them up.

Preferably, a backing layer, in particular an occlusive backing layer, is based on a polymer selected from the group consisting of polyolefins, olefin copolymerisates, polyesters, copolyesters, polyamides, copolyamides, polyurethanes and the like. Examples of suitable materials which may be cited are polyesters, among them polyethylene terephthalates (PET) in particular, as well as polycarbonates, polyolefins such as, for example, polyethylenes, polypropylenes or polybutylenes, polyethylene oxides, polyurethanes, polystyrenes, polyamides, polyimides, polyvinyl acetates, polyvinyl chlorides, polyvinylidene chlorides, copolymerisates such as, for example, acrylonitrile-butadiene-styrene terpolymers, or ethylene-vinyl acetate-copolymerisates. A preferred material for a backing layer is selected from a polyester, particular preferably from a polyethylene terephthalate. A backing layer of this type may, for example, be obtained from 3M (USA) under the trade name Scotchpak 1109.

In addition, a backing layer for a TDS in accordance with the invention may comprise a covering layer or overtape, which protrudes laterally beyond the edges of the at least one active ingredient-containing matrix on a non-aqueous basis and thus can permit better adhesion of the TDS in accordance with the invention to the skin. A preferred covering layer or a preferred overtape here is occlusive in configuration.

The overtape may be multi-layered in configuration. In particular, an overtape comprises a layer of adhesive which is free from active ingredient and an overtape film, wherein an overtape film preferably comprises a polymer selected from the group formed by polyolefins, olefin copolymerisates, polyesters, copolyesters, polyamides, copolyamides, polyurethanes and the like. Examples of suitable materials that may be cited are polyesters, and of these, polyethylene terephthalates in particular, as well as polycarbonates, polyolefins such as, for example, polyethylenes, polypropylenes or polybutylenes, polyethylene oxides, polyurethanes, polystyrenes, polyamides, polyimides, polyvinyl acetates, polyvinyl chlorides, polyvinylidene chlorides, copolymerisates such as, for example, acrylonitrile-butadiene-styrene terpolymers, or ethylene-vinyl acetate-copolymerisates. A preferred material for an overtape is selected from a polyester, particularly preferably a polyethylene terephthalate.

If a transdermal application system does not comprise an overtape, or an overtape serves only for the protection of the active ingredient-containing matrix against cold flow, a preferred matrix for the transdermal delivery system in accordance with the invention is configured to be adhesive at least on its application side. This guarantees continuous contact of the application side with the skin, and thus a continuous delivery of active ingredient on or through the skin of a user.

In one embodiment of the TTS in accordance with the invention, as mentioned above, the active ingredient-containing matrix or an application layer containing an active ingredient or free from active ingredient may be covered with a removable protective film which is also known in the art as a release liner. When storing the TTS, then, the active ingredient-containing matrix or the application layer is effectively protected from mechanical influences and/or from the unwanted ingress of air. By preventing the unwanted ingress of air, decomposition of active ingredients contained in the matrix, in particular of oxygen-sensitive active ingredients, is prevented and thus the storage or long-term stability of the patch (hereinafter, the TTS will also be termed a patch) is improved. In order to apply the patch, prior to attaching the system to the skin, the protective film of the patch is first removed. For better grip and in order to facilitate removal of the protective film in this context, in advantageous patches, the protective film protrudes beyond the edge of the remaining patch.

As already mentioned, inventive embodiments of a transdermal therapeutic system comprise an active ingredient matrix, preferably on a non-aqueous basis, containing at least one active ingredient. In accordance with a preferred embodiment, the active ingredient is in the form of a solution in the dried matrix or is dispersed in the polymer or the polymer blend forming the matrix. In accordance with a further preferred embodiment, the active ingredient is also present in a matrix which has not been dried, i.e., for example, in a coating solution containing an active ingredient, in the form of a solution.

The term “solution” in the context of the present invention should be understood here to mean a mixture of a solvent and a solvate, wherein the solvate is molecularly dispersed, i.e. may have a particle size of less than 1 nm. Furthermore, a solution may also have colloidally dispersed dissolved particles with a dimension in the range 1 nm to 1 μm and/or coarsely dispersed dissolved particles with a dimension of more than 1 μm.

In accordance with a preferred method, therefore, the active ingredient used for the production of the active ingredient-containing matrix is used in the form of a solution. In particular in this regard, a solution of active ingredient is used which has previously been dried in order to reduce the water content of the solution of the active ingredient. Drying of this type may preferably be carried out at a temperature of at least approximately 60° C. and of at most approximately 70° C., preferably over a period of at least approximately 1 hour and of at most approximately 5 hours, so that the dried active ingredient solution advantageously has a water content in the range from less than approximately 0.5% by weight, particularly preferably from less than approximately 0.2% by weight, in particular of less than approximately 0.1% by weight, particularly preferably of less than approximately 0.05% by weight. Suitable methods for determining the water content are known to the person skilled in the art. As an example, a determination of the water content may be made by means of a Karl-Fischer titration.

In this manner, with the aid of the method in accordance with the invention, a preferred active ingredient-containing matrix, preferably on a non-aqueous basis, and/or a laminate and/or a transdermal therapeutic system, in particular a transdermal therapeutic system, may be produced which has a water content, in the dried and temperature-conditioned matrix, of up to approximately 2% by weight, preferably of up to approximately 1% by weight, particularly preferably of up to approximately 0.75% by weight, in particular of up to approximately 0.5% by weight, particularly preferably of up to approximately 0.2% by weight (measured using Karl-Fischer titration).

As a function of the desired delivery rate, application period and/or use, as mentioned, a preferred TDS in accordance with the invention may comprise more than one active ingredient-containing matrix, preferably on a non-aqueous basis, wherein the active ingredients employed may be identical or different and may be present in different concentrations.

Thus, for example, a first matrix layer of a preferred TDS in accordance with the invention may contain a hormone from the group formed by estrogens and a second or further matrix layer may contain a hormone from the progestogen group. Clearly, active ingredients of different categories such as, for example an antiemetic, may be used in a first matrix layer and an opioid in a second or further matrix layer of the TTS in accordance with the invention. Alternatively, for example, an antiemetic such as scopolamine may be contained in a first matrix layer and a caffeine in a second matrix layer.

However, in accordance with a particularly preferred embodiment, the TTS in accordance with the invention contains only one active ingredient which is preferably selected from an antiemetic, particularly preferably a tropane alkaloid, in particular scopolamine.

A particularly preferred embodiment herein comprises a first scopolamine-containing matrix on a non-aqueous basis and a second scopolamine-containing matrix on a non-aqueous basis, wherein particularly preferably, the first and the second matrix layers are separated from each other by means of a control membrane.

In particular, the present invention in this regard encompasses a transdermal therapeutic system for use in the treatment of travel sickness. Preferably, the treatment of travel sickness in this regard is carried out over an application period from one day up to seven days, particularly preferably of up to three days.

Further particularly suitable active ingredients are selected from the dopamine agonist class, particular preferably from the class of D2 agonists and ergot alkaloid derivatives, in particular from rotigotine, pramipexole, ropinirole, cabergoline and/or lisuride. A transdermal therapeutic system of this type is in particular used for application in the treatment of Parkinson's disease, idiopathic restless leg syndrome, amenorrhea, acromegaly and hyperprolactinemia.

Further particularly suitable active ingredients include anti-dementia drugs such as, for example, rivastigmine, donezepil, galantamine, memantine, in particular rivastigmine, wherein a TDS of this type can be used for the treatment of dementia diseases, in particular for the treatment of brain disorders such as problems with memory and concentration and thought processes. However, such TDSs may also be employed when the full-on development of dementia arises with changes to the personality such as distrust, anxiety or depression. In particular, the TDSs may be used for the treatment of Alzheimer's disease.

Finally, particularly suitable active ingredients also include the class of analgesics and sedatives, particularly preferably opioids such as, for example, buprenorphine, fentanyl, sufentanil, alfentanil and remifentanil, wherein a TDS of this type is preferably used for the treatment of pain and/or analgesics.

In this regard, further preferred active ingredients which may be contained in the TDS in accordance with the invention are selected from:

1. Cardioactive medication, for example organic nitrates, such as nitroglycerin, isosorbide dinitrate and isosorbide mononitrate, quinidine sulphate, procainamide, thiazides such as bendroflumethiazide, chlorothiazide and hydrochlorothiazide, nifedipine, nicardipine, adrenergic blockers such as timolol and propranolol, verapamil, diltiazem, captopril, clonidine and prazosine;

2. Androgenic steroids such as testosterone, methyltestosterone and fluoxymesterone;

3. Estrogens such as conjugated estrogens, esterified estrogens, estropipate, 17β-estradiol, 17β-estradiol valerate, equilin, mestranol, estrone, estriol, 17β-ethinylestradiol and diethylstilbestrol;

4. Progestogens such as progesterone, 19-norprogesterone, norethindrone, norethindrone acetate, chlormadinone, ethisterone, etonogestrel, medroxyprogesterone acetate, hydroxyprogesterone caproate, norethynodrel, norelgestromin, 17α-hydroxyprogesterone, dydrogesterone, dimethisterone, ethinylestrenol, norgestrel, demegestone, promegestone and megestrolacetate;

5. Medication which acts on the central nervous system, for example sedatives, hypnotics, anxiolytics, analgesics and anesthetics such as naloxone, haloperidol, fluphenazine, pentobarbital, phenobarbital, secobarbital, codeine, lidocaine, tetracaine, dibucaine, cocaine, procaine, mepivacaine, bupivacaine, etidocaine, prilocaine, benzocaine, tapentadol and nicotine;

6. Nutrients and food supplements such as vitamins, essential amino acids and essential fats;

7. Anti-inflammatory agents such as hydrocortisone, cortisone, dexamethasone, fluocinolone, triamcinolone, prednisolone, flurandrenolide, methylprednisolone, prednisone, methylprednisolone, corticosterone, paramethasone, betamethasone, ibuprofen, naproxen, fenoprofen, fenbufen, flurbiprofen, ketoprofen, suprofen, indomethacin, piroxicam, aspirin, salicylic acid, diflunisal, methyl salicylate, phenylbutazone, sulindac, mefenaminic acid, tolmetin and the like.

8. Antihistamines such as diphenhydramine, dimenhydrinate, perphenazine, triprolidine, pyrilamine, chlorcyclizine, promethazine, carbinoxamine, tripelennamine, brompheniramine, clorprenaline, terfenadine and chlorpheniramine;

9. Respiratory agents such as theophylline and β-adrenergic agonists such as albuterol, terbutaline, metaproterenol, ritodrine, carbuterol, fenoterol, quinterenol, rimiterol, salmefamol, solerenal and tetroquinol;

10. Sympathomimetics and parasympathomimetics such as dopamine, norepinephrine, phenylpropanolamine, phenylephrine, physostigmine, pseudoephedrine, amphetamine, propylhexedrine and epinephrine;

11. Myotics such as pilocarpine and the like;

12. Cholinergic agonists such as choline, acetylcholine, methacholine, carbachol, bethanechol, pilocarpine, muscarine and arecoline;

13. Antimuscarinic or muscarinic cholinergic antagonists such as atropine, methscopolamine, homatropine methylbromide, methantheline, cyclopentolate, tropicamide, propantheline, dicyclomine and eucatropine;

14. Mydriatics such as atropine, cyclopentolate and hydroxyamphetamine;

15. Psychoanaleptics such as 3-(2-aminopropyl)indole, 3-(2-aminobutyl)indole and the like;

16. Anti-infectives such as antibiotics, including penicillin, tetracycline, chloramphenicol, sulfacetamide, sulfamethazine, sulfadiazine, sulfamerazine, sulfamethizole and sulfisoxazole; antiviral agents; antibacterial agents such as erythromycin and clarithromycin, and other anti-infectives including nitrofurazone and the like;

17. Dermatological agents such as vitamin A and vitamin E;

18. Hormone-like substances such as natural and synthetic prostaglandins, for example PGE1, PGE2a and PGF2a and the PG analog misoprostol;

19. Antispasmodics such as atropine, methantheline, papaverine and methscapolamine;

20. Antidepressives such as isocarboxazide, phenelzine, tranylcypromine, imipramine, amitriptyline, trimipramine, doxepine, desipramine, nortriptyline, protriptyline, amoxapine, maprotiline and trazodone;

21. Antidiabetics such as insulin, and cancer medication such as tamoxifen and methotrexate;

22. Anorectics such as dextroamphetamine, methamphetamine, phenylpropanolamine, fenfluramine, diethylpropion, mazindole and phentermine;

23. Antiallergics such as antazoline, methapyrilene, chlorpheniramine, mizolastine, pyrilamine and pheniramine;

24. Sedatives such as reserpine, chlorpromazine and anxiolytic benzodiazepines such as alprazolam, chlordiazepoxide, clorazepate, halazepam, oxazepam, prazepam, flurazepam, triazolam, lorazepam and diazepam;

25. Antipsychotics such as thiopropazate, chlorpromazine, triflupromazine, mesoridazine, piperacetazine, thioridazine, acetophenazine, fluphenazine, perphenazine, trifluoperazine, chlorprothixene, thiothixene, haloperidol, bromperidol, loxapine and molindone;

26. Decongestants such as phenylephrine, ephedrine, naphazoline, tetrahydrozoline;

27. Antipyretics such as acetyl salicylic acid, salicylamide and the like;

28. Antimigraine agents such as dihydroergotamine and pizotyline;

29. Medication for the treatment of nausea and sickness such as chlorpromazine, granisetron, perphenazine, prochlorperazine, promethazine, thiethylperazine, triflupromazine and trimeprazine;

30. Antimalarial agents such as 4-aminoquinoline, a-aminoquinoline, chloroquine and pyrimethamine;

31. Ulcer treatment agents such as misoprostol, omeprazole and enprostil;

32. Peptides such as growth hormone releasing factor;

33. Medicaments for Parkinson's disease, spasticity and acute muscle spasms such as levodopa, carbidopa, amantadine, apomorphine, brorocriptine, selegiline (deprenyl), trihexyphenidyl hydrochloride, benztropine mesylate, procyclidine hydrochloride, baclofen, diazepam and dantrolene;

34. Antiestrogen or hormonal agents such as tamoxifen or human chorionic gonadotropin;

35. Aromatase inhibitors such as anastrozole;

36. Cholinesterase inhibitors such as physostigmine or pyridostigmine;

The quantity of the active ingredient which ought to be contained in the composition varies as a function of the specific active ingredient, the desired therapeutic effect and the time period during which the TDS is supposed to be used for a therapy. In most active ingredients, penetration through the skin is the rate determining step for their delivery. The quantity of the active ingredient and the release rate are thus typically selected in a manner such that transdermal delivery is obtained which over a longer time period is characterized by a time dependency which is essentially of zero order.

Preferably, the quantity of the active ingredient in the system can vary from at least approximately 0.3% by weight, preferably at least approximately 1% by weight, particularly preferably at least approximately 2.5% by weight, in particular from at least approximately 5% by weight and/or up to approximately 50% by weight, preferably up to approximately 30% by weight, particularly preferably up to approximately 20% by weight, in particular up to 10% by weight.

In order to construct an active ingredient-containing matrix, preferably on a non-aqueous basis or an active ingredient-free layer of the TTS, any of the materials which are usually suitable for transdermal therapeutic systems are suitable. Preferably, for example, tackifiers may be added to a matrix in order to obtain a self-adhesive (i.e. adhesive) active ingredient-containing matrix, as an alternative or in addition to the aforementioned skin side adhesive layer. In addition, the matrix may also be constructed from a self-adhesive polymer. In this regard, specific mention may be made of polymers which are used in the production of transdermal systems and which are physiologically harmless such as, for example, polyisobutylene, homopolymers and copolymers of (meth)acrylates, polyvinylethers, polyisoprene rubbers, styrene-butadiene copolymers or styrene-butadiene-styrene copolymers and silicones. Examples of (meth)acrylate copolymers that may be mentioned are copolymers of alkylacrylates and/or alkylmethacrylates and further unsaturated monomers such as acrylic acid, methacrylic acid, acrylamide, dimethylacrylamide, dimethylaminoethylacrylamide, acrylonitrile and/or vinyl acetate.

Preferably, what are known as solubilizers or cosolvents and/or permeation enhancers may also be used such as, for example, from the group formed by alcohols, preferably from the group formed by aliphatic alcohols with a terminal OH group, in particular with a chain length between 10 and 14 carbon atoms, particularly preferably dodecanol. Further preferred cosolvents and/or permeation enhancers may be selected from lauryl lactate, vitamin E, aloe vera oil, propylene glycol monolaureate and/or from the group formed by propyl esters, in particular from myristic acid isopropyl esters.

In particular, a content of one or more of the preferred cosolvents and/or permeation enhancers causes a controlled and constant delivery of active ingredient from the preferred transdermal therapeutic system, in particular from a transdermal therapeutic system comprising an active ingredient from the class of dopamine agonists, particularly preferably from the class of D2 agonists and ergot alkaloid derivatives, in particular from rotigotine, pramipexol, ropinirole, cabergoline and/or lisuride, particularly preferably rotigotine. Preferably, a content of a cosolvent or a permeation enhancer in the active ingredient-containing matrix on a non-aqueous basis is at least approximately 1% by weight, in particular at least approximately 5% by weight, particularly preferably at least approximately 7% by weight and/or preferably up to approximately 20% by weight, in particular up to approximately 10% by weight, particularly preferably up to approximately 8% by weight.

In principle, a suitable basis weight for the dried active ingredient-containing matrix is in the range which is usual for transdermal therapeutic systems. However, a preferred basis weight for an at least one active ingredient-containing matrix, preferably on a non-aqueous basis, is at least approximately 10 mg/10 cm², particularly preferably at least approximately 20 mg/10 cm², in particular at least approximately 30 mg/10 cm², particularly preferably at least approximately 35 mg/10 cm² and/or preferably up to approximately 100 mg/10 cm², particularly preferably up to approximately 80 mg/10 cm², in particular up to approximately 70 mg/10 cm², particularly preferably up to approximately 65 mg/10 cm².

When the TDS in accordance with the invention has more than one matrix, then the basis weight of a first or further matrix is preferably at least approximately 30 mg/10 cm², particularly preferably at least approximately 35 mg/10 cm², in particular at least approximately 40 mg/10 cm². At most, the preferred basis weight of a first or a further matrix is up to approximately 70 mg/10 cm², particularly preferably up to approximately 65 mg/10 cm², in particular up to approximately 60 mg/10 cm². However, in this regard, a first or further matrix does not have to have the same basis weight; rather, a first matrix may, for example, have a basis weight of at least approximately 50 mg/10 cm² up to approximately 65 mg/10 cm², and a further matrix may have a basis weight of, for example, at least approximately 35 mg/10 cm² up to approximately 55 mg/10 cm².

Tailoring of the basis weight to the total layer thickness or the total basis weight of the TDS in accordance with the invention appears to be particularly useful when a first initial delivery of an active ingredient from an application layer with one active ingredient content has to be matched to a second, longer-lasting active ingredient delivery from a matrix layer facing the backing layer. Similarly, when selecting the basis weight, the influence on the support properties of the TDS should be taken into consideration.

In order to keep the moisture content, in particular the water content, in the active ingredient-containing matrix of the TDS in accordance with the invention as low as possible, in accordance with a preferred method for the production of the laminate, a backing layer and/or a protective film with moisture-absorbing material properties such as, for example using materials comprising a silica gel formulation, may be used.

Alternatively or additionally, a preferred method in accordance with the invention proposes the use of a pre-dried packaging or a packaging material, in particular a pouch, in which the TDS is packaged, which furthermore may be provided with moisture-absorbing materials.

A moisture-absorbing material such as, for example, the material available from CSP Technologies with the trade mark Activ-Film™, may also be introduced into the pouch in a packaging step, whereupon the moisture content, in particular the water content, in the environment of the packaged TDS during storage can be additionally reduced. Clearly, a pouch already provided with a moisture-absorbing material could be used directly for packaging of the TDS.

The process of drying and/or temperature-conditioning of the active ingredient-containing matrix may preferably take place under a nitrogen atmosphere, in order to minimize the ingress of oxygen onto or into the active ingredient-containing matrix and thus to prevent any possible oxidation and/or recrystallization of the active ingredient. Alternatively or in addition, oxygen and/or carbon dioxide-absorbing materials may also be employed.

Further features of the invention will become apparent from the present description of exemplary embodiments in combination with the claims. The individual features in one embodiment in accordance with the invention can be employed individually or in combination and do not limit the scope of protection of the present invention.

### EXAMPLES

Examples 1 and 2 below describe the production of transdermal therapeutic systems in accordance with the invention, as well as stability studies.

### Example 1

### Exemplary Embodiment Concerning Production, and Stability Study of a Scopolamine TDS in Accordance with the Invention

A solution of 40.3% by weight of polyisobutylene (PIB) adhesive (Oppanol B80, from BASF), 25.8% by weight of PIB adhesive (Oppanol B10, from BASF), 20% by weight of mineral oil (paraffin Ph. Eur., Klearol, from Sonneborn), 5% by weight of silicon dioxide (silicon dioxide Ph.Eur., high purity and amorphous high dispersity silicic acid, hydrophilic, Aerosil 200 Pharma, from Evonik), 9% by weight of scopolamine base (hyoscine, from Alkaloids Private Limited) and a suitable quantity of chloroform was coated onto a polyethylene terephthalate (PET) film siliconized on one side (Primeliner PET 75 μm 1S, from Loparex) in a manner such that after drying, a matrix layer with a basis weight of approximately 56 g/m² was produced. In order to remove the solvent, drying was carried out at 60° C. for 15 min. The matrix layer was laminated with a polyester protective film (from Mitsubishi) siliconized on one side.

A solution of 33.8% by weight of PIB adhesive (Oppanol B80, from BASF), 21.6% by weight of PIB adhesive (Oppanol B10, from BASF), 36.3% by weight of mineral oil (paraffin Ph. Eur., Klearol, from Sonneborn), 5% by weight of silicon dioxide (silicon dioxide Ph.Eur., high purity and amorphous high dispersity silicic acid, hydrophilic, Aerosil 200 Pharma, from Evonik), 3.3% by weight of scopolamine base (hyoscine, from Alkaloids Private Limited) and a suitable quantity of chloroform was coated onto a polyethylene terephthalate (PET) film siliconized on one side in a manner such that after drying, an application layer (=adhesive layer) with a basis weight of approximately 45 g/m² was produced. In order to remove the solvent, drying was carried out at 60° C. for 15 min.

A microporous polypropylene membrane (thickness 25 □m; microporous (porosity 41%), from Celgard) soaked with mineral oil (paraffin Ph. Eur., Klearol, from Sonneborn) was laminated onto an application layer. The PET film was removed from the matrix layer and the matrix layer was applied to the laminate formed by the application layer and membrane.

The determination of the water content of the active ingredient-containing matrix on a non-aqueous basis was carried out using Karl Fischer titration and yielded a 0.24% by weight water content.

The TDS obtained in this manner was then treated as follows:
T1: the punched and pouch-packaged TDSs were temperature-conditioned within 24 h following its production, for 24 h at 75° C.;
T2: no temperature-conditioning of the punched and pouch-packaged TDSs was carried out;

The transdermal therapeutic systems were stored at room temperature (RT) and at 40° C./75% r.h. (relative humidity) respectively. The stability studies show that within the test period of six months in the TDS in accordance with the invention, which was temperature-conditioned 24 hours after its production, no crystals of active ingredient had formed. In contrast to this, in TDSs which had not been temperature-conditioned, crystals of active ingredient could be observed even after 1.5 weeks (see Table 1).

TABLE 1
Stability studies regarding the crystallization
of active ingredient in temperature-conditioned
(T1) and non-temperature-conditioned (T2) TDS.

| | | T1 (the pouch- | |
|---|---|---|---|
| | | packaged TDSs were | |
| Duration of storage | | temperature-conditioned | T2 (the pouch- |
| (respectively at | | within 24 h following | packaged TDSs were |
| room temperature | | production for 24 h | not temperature- |
| and at 40° C.) | | at 75° C.) | conditioned) |
| 1.5 | Weeks | No crystals | Onset of |
| | | | crystallization |
| 1 | month | No crystals | Crystals |
| 2 | months | No crystals | Crystals |
| 3 | months | No crystals | Crystals |
| 6 | months | No crystals | Crystals |

### Example 2

A solution of 26.7% by weight of polyisobutylene (PIB) adhesive (Oppanol N80, from BASF), 33.3% by weight of PIB adhesive (Oppanol B10, from BASF), 27% by weight of mineral oil (paraffin Ph.Eur., Klearol, from Sonneborn), 5% by weight of silicon dioxide (silicon dioxide Ph.Eur., high purity and amorphous high dispersity silicic acid, hydrophilic, Aerosil 200 Pharma, from Evonik), 8% by weight of scopolamine base (hyoscine, from Alkaloids Private Limited) and a suitable quantity of 2-propanol and heptane was coated onto a polyethylene terephthalate (PET) film siliconized on one side (Primeliner PET 75 μm 1S, from Loparex) in a manner such that after drying, a matrix layer with a basis weight of approximately 56 g/m² was produced. Drying was carried out for 30 min at 75° C. in order to remove the solvent. The matrix layer was laminated with a polyester protective film siliconized on one side (polyester film from Mitsubishi).

A solution of 24.3% by weight of PIB adhesive (Oppanol N80, from BASF), 30.4% by weight of PIB adhesive (Oppanol B10, from BASF), 37% by weight of mineral oil (paraffin Ph.Eur., Klearol, from Sonneborn), 5% by weight of silicon dioxide (silicon dioxide Ph.Eur., high purity and amorphous high dispersity silicic acid, hydrophilic, Aerosil 200 Pharma, from Evonik), 3.3% by weight of scopolamine base (hyoscine, from Alkaloids Private Limited) and a suitable quantity of 2-propanol and heptane was coated onto a polyethylene terephthalate (PET) siliconized on one side in a manner such that after drying, an application layer (=adhesive layer) with a basis weight of approximately 45 g/m² was produced.

Drying was carried out for 30 min at 75° C. in order to remove the solvent. A microporous polypropylene membrane (thickness 25 □m; microporous (porosity 41%) from Celgard) soaked with mineral oil (paraffin Ph.Eur., Klearol, from Sonneborn) was laminated onto the application layer. The PET film was removed from the matrix layer and the matrix layer was applied to the laminate formed by the application layer and membrane. Next, the laminate was punched in order to obtain transdermal therapeutic systems (TTS) which were subsequently packaged into pouches.

The determination of the water content of the active ingredient-containing matrix on a non-aqueous basis was carried out using Karl Fischer titration and yielded a 0.24% by weight water content.

Studies on the Crystallization of the Active Ingredient Scopolamine:

The pouch-packaged TTSs obtained in this manner were temperature-conditioned for 30 minutes at 75° C. in a drying cabinet 24 hours following production and stored for a period of 6 months at a temperature of 25° C./60 r.h. Tests regarding the crystallization of the active ingredient scopolamine showed that within the test period of 6 months, no active ingredient crystals had formed in any of the TDSs in accordance with the invention.

Studies on the Chemical Stability of the Active Ingredient Scopolamine:

Furthermore, studies regarding the chemical stability of the active ingredient were carried out. In a first step, how the duration of temperature-conditioning at an exemplary temperature of 75° C. affected the chemical stability of the active ingredient or the formation of contaminants was tested.

In this regard, the pouch-packaged TDSs were temperature-conditioned for one hour, 5.5 hours, 13.5 hours or 24 hours at a temperature of 75° C. Next, the active ingredient-containing matrix was tested as regards contaminants which resulted from a degradation of the active ingredient. The results of the tests are shown in Table 2.

Table 2 shows that a temperature-conditioning duration of beyond approximately 13.5 hours caused an increase in the profile of the contaminants, in the present case of the contaminants “B” and “C”. A temperature-conditioning duration of approximately 1 hours and approximately 5.5 hours, on the other hand, exhibited only a small influence on the degradation of the active ingredient.

TABLE 2
Stability of active ingredient in pouch-packaged TDSs as a
function of the duration of the temperature-conditioning.

| | Temperature-conditioning (75° C.) | | | | |
|---|---|---|---|---|---|
| | 0 h | 1 h | 5.5 h | 13.5 h | 24 h |
| Active ingre- | 1.47 | 1.45 | 1.45 | 1.49 | 1.38 |
| dient content | | | | | |
| (mg/TDS) | | | | | |
| Purity (%) | | | | | |
| VU A | n.d. | n.d. | n.d. | n.d. | n.d. |
| VU B | n.d. | n.d. | n.d. | 0.30 | 0.77 |
| VU C | 0.10 | 0.12 | 0.13 | 0.13 | 0.16 |
| VU D | n.d. | n.d. | n.d. | n.d. | n.d. |

VU = contaminants;
n.d. = not detected;

In a subsequent step, TDSs which had been temperature-conditioned for 30 minutes or for 5 hours at a temperature of 75° C. were each stored for 6 months at a temperature of 25° C./60% r.h. The tests also showed that over the entire test period of 6 months, there was a slight degradation of the active ingredient when the TDS was temperature-conditioned at temperature for 30 minutes or for 5 hours at a temperature of 75° C. (see Tables 3A and 3B).

TABLE 3A
Stability of active ingredient in temperature-conditioned
TDSs after storage for 6 months at 25° C./60% r.h,
with temperature-conditioning at 75° C. for 30 minutes.

| | Temperature-conditioning | | |
|---|---|---|---|
| | (75° C. for 30 min.) | | |
| | Start | 3 M 25° C. | 6 M 25° C. |
| Content % | 106.0 | 100.7 | 100.0 |
| Purity % | | | |
| VU A | n.d. | n.d. | n.d. |
| VU B | n.d. | <RL | 0.22 |
| VU C | n.d. | <RL | <RL |
| VU D | n.d. | n.d. | n.d. |

VU = contaminants;
RL = reporting level (0.1%);
n.d. = not detected;

TABLE 3B
Stability of active ingredient in temperature-conditioned
TDSs after storage for 6 months at 25° C./60% r.h.,
with temperature-conditioning at 75° C. for 5 hours.

| | Temperature-conditioning | | |
|---|---|---|---|
| | (75° C. for 5 hours) | | |
| | Start | 3 M 25° C. | 6 M 25° C. |
| Content % | 99.3 | 102.0 | 102.0 |
| Purity % | | | |
| VU A | n.d. | n.d. | n.d. |
| VU B | n.d. | 0.14 | 0.23 |
| VU C | n.d. | <RL | <RL |
| VU D | n.d. | n.d. | n.d. |

Finally, it should be pointed out once again that the examples described above in detail relate solely to exemplary embodiments which can be modified by the person skilled in the art in a wide variety of manners without departing from the scope of the invention. Furthermore, the use of the indefinite article “a” or “an” does not exclude that the features concerned could also be present as a plurality thereof.

1. A method for the production of a transdermal delivery system, comprising the steps of:
(i) providing at least one active ingredient-containing matrix, preferably an active ingredient-containing matrix on a non-aqueous basis,
(ii) applying the at least one active ingredient-containing matrix to a film in order to obtain a laminate with an active ingredient-containing matrix,
(iii) optionally, drying the laminate comprising the active ingredient-containing matrix,
(iv) optionally, punching out transdermal therapeutic systems in order to obtain laminar active ingredient cores,
(v) optionally, packaging transdermal therapeutic systems,
(vi) temperature-conditioning the active ingredient-containing matrix on a non-aqueous basis and/or the laminate and/or the punched transdermal therapeutic systems and/or the packaged transdermal therapeutic systems,
wherein the temperature-conditioning is carried out directly, preferably within 7 days, preferably within three days, particularly preferably within two days, in particular within 24 hours following application of the at least one active ingredient-containing matrix to the film.

2. The method for the production of a transdermal delivery system as claimed in claim 1, wherein the temperature-conditioning is carried out at a temperature of at least approximately 50° C., preferably at a temperature of at least approximately 60° C., particularly preferably at a temperature of at least approximately 70° C., and/or at a temperature of up to approximately 120° C., preferably at a temperature of up to approximately 100° C., particularly preferably at a temperature of up to approximately 90° C., in particular at a temperature of up to approximately 80° C., particularly preferably at a temperature of approximately 75° C.

3. The method for the production of a transdermal delivery system as claimed in claim 1, wherein the temperature-conditioning is carried out over a time period of up to 12 hours, preferably over a time period of up to 6 hours, particularly preferably over a time period of up to 2.5 hours, in particular over a time period of up to approximately one hour, particularly preferably over a time period of up to approximately 30 minutes, more particularly preferably over a time period of up to approximately 15 minutes.

4. A transdermal delivery system, wherein the transdermal delivery system is obtainable by means of a method as claimed in claim 1.

5. The transdermal delivery system as claimed in claim 4, wherein the transdermal delivery system furthermore has an adhesive application layer which is free from active ingredient or contains an active ingredient.

6. The transdermal delivery system as claimed in claim 4, which has a control membrane between the matrix, preferably the matrix on a non-aqueous basis, and the adhesive application layer which is free from active ingredients or which contains an active ingredient.

7. The transdermal delivery system as claimed in claim 4, wherein at least one matrix on a non-aqueous basis comprises a mineral oil.

8. The transdermal delivery system as claimed in claim 4, wherein at least one matrix, preferably the matrix on a non-aqueous basis, comprises a silicon dioxide.

9. The transdermal delivery system as claimed in claim 4, wherein the matrix, preferably the matrix on a non-aqueous basis, comprises at least one polymer, preferably at least two polymers, which are selected from the group formed by polybutylenes, in particular by polyisobutylenes.

10. The transdermal delivery system as claimed in claim 9, wherein one of the at least two polybutylenes, in particular polyisobutylenes, has a molecular weight of at least approximately 20,000 g/mol and/or at most approximately 100,000 g/mol, and/or a second polybutylene, in particular a polyisobutylene, has a molecular weight of at least approximately 500,000 g/mol and/or at most approximately 3,500,000 g/mol.

11. The transdermal delivery system as claimed in claim 4, which has an occlusive backing layer.

12. The transdermal delivery system as claimed in claim 4, for medical, veterinary or cosmetic use.

13. The transdermal delivery system as claimed in claim 4, comprising an antiemetic, a dopamine agonist, an analgesic, a sedative and/or an anti-dementivum.

14. The transdermal delivery system as claimed in claim 13, wherein the antiemetic is selected from a tropane alkaloid, in particular scopolamine, and wherein the dopamine agonist is selected from a D2 agonist, in particular rotigotine, and wherein the analgesic is selected from buprenorphine and/or fentanyl, and wherein the anti-dementivum is selected from rivastigmine.

15. A transdermal delivery system as claimed in claim 4, for use in the treatment of travel sickness, Parkinson's disease and/or restless leg syndrome, pain, in particular cancer-related pain, and Alzheimer's disease.
